(19) United States
(12) Patent Application Publication
(10) Pub. No.: US 2013/0210102 A1
(43) Pub. Date: Aug. 15, 2013

(54) METHODS FOR DETOXIFYING A LIGNOCELLULOSIC HYDROLYSATE

(71) Applicant: BP Corporation North America Inc., (US)

(72) Inventors: Malgorzata Slupska, San Diego, CA (US); Yukiko Sato, San Diego, CA (US); Karen Kustedjo, San Diego, CA (US); Kelvin Wong, San Diego, CA (US)

(73) Assignee: BP Corporation North America Inc., Houston, TX (US)

(21) Appl. No.: 13/764,943

(22) Filed: Feb. 12, 2013

Related U.S. Application Data
(60) Provisional application No. 61/597,936, filed on Feb. 13, 2012.

(51) Int. Cl. C12P 7/10

(52) U.S. Cl. CPC C12P 7/10
USPC 435/165; 252/182.31

(57) ABSTRACT

The present disclosure relates to methods for detoxifying a hydrolysate obtained from a lignocellulosic biomass and methods of producing ethanol from the detoxified hydrolysate. The present methods provide detoxified hydrolysates in which the quantity of compounds that are deleterious to fermenting microorganisms are substantially reduced relative to the starting hydrolysate and in which the amount of total fermentable sugars loss is minimal.

### 1. BACKGROUND

Many industrial products are produced by microorganisms grown in culture. Microorganism growth may be supported by soluble sugar molecules released by lignocellulosic biomasses. Lignocellulosic biomasses consist primarily of cellulose (polymers of glucose linked by β-1,4-glucosidic bonds), hemicellulose (polysaccharide composed of different five (C5)-carbon sugars and six (C6)-carbon sugars linked by variety of different β and α linkages) and lignin (complex polymer consisting of phenyl propane units linked by ether or carbon-carbon bonds). In some cases, lignocellulosic biomasses are subject to dilute acid hydrolysis during which hemicellulose is hydrolyzed to monomeric sugars (liquid stream) and the crystalline structure of cellulose is damaged, facilitating future enzymatic digestion (solid fiber). The liquid containing C5 and C6 sugars, so called hydrolysate, is separated from cellulose and lignin solids and can be fermented to various products such as ethanol. In addition to sugars however, hydrolysate also contains aliphatic acids, esters (acetate), phenolics (different compounds obtained from lignin hydrolysis) and products of sugar dehydration, including the furan aldehydes furfural and 5-hydroxymethyl furfural (5-HMF). Most of these compounds have a negative impact on microorganisms and can inhibit fermentation. Detoxification of the hydrolysate prior to fermentation is one measure that can be taken in order to avoid inhibition caused by toxic compounds present in the hydrolysate.

Various methods of detoxification have been tested, with alkaline overliming being efficient and cost effective. During the overliming process, the pH of the hydrolysate is temporarily raised, usually at an elevated temperature, from a pH of approximately 2 to a pH of between 9 and 10 through the addition of an appropriate amount of calcium hydroxide (lime). After some time, typically about 30 minutes, the pH of the hydrolysate solution is lowered through the addition of acid to a pH suitable for fermenting microorganisms. In the detoxification process, furan aldehydes are degraded and acids (mineral and organic) are neutralized.

Overliming has been known for a long time (Leonard and Hajny, 1945, Ind. Eng. Chem., 37 (4):390-395) and still is considered an efficient detoxification method. However, a significant drawback of the method is the considerable amount of loss of fermentable sugars that occurs during detoxification. See, e.g., Larsson et al., 1999, Appl. Biochem. Biotechnol. 77-79:91-103. The loss of fermentable sugars results in lower overall yields of fermentable products such as ethanol. In addition, the formation of insoluble calcium sulfate (gypsum) during detoxification is problematic. See, e.g., Martinez et al., 2001, Biotechnol. Prog. 17(2):287-293. Gypsum formation causes fouling and pipeline clogging, which significantly drive up maintenance costs. To overcome problems associated with calcium hydroxide, other bases have been attempted for the purpose of hydrolysate detoxification, which have met with varying levels of success. See, e.g., Alriksson et al., 2005, Appl. Biochem. Biotechnol. 121-124:911-922.

Accordingly, there is a need for new and improved processes to reduce fermentation inhibitors and detoxify hydrolysates obtained from lignocellulosic biomasses. In particular, there is a need for detoxification processes that are economically viable and provide detoxified hydrolysates capable of producing high yields of ethanol.

### 2. SUMMARY

The present disclosure stems from the discovery that a multiple step detoxification process can substantially reduce the amounts of compounds in a hydrolysate obtained from a lignocellulosic biomass (sometimes referred to herein as a “lignocellulosic hydrolysate”) that are harmful to a fermenting microorganism, and that the detoxification process results in minimal losses of fermentable sugars. As used herein, the term “detoxification” refers to a process in which one or more compounds that are detrimental to a fermenting microorganism (referred to herein as “toxins”) are removed from a starting lignocellulosic hydrolysate or inactivated, thereby forming a detoxified hydrolysate. As used herein, the phrase “detoxified hydrolysate” refers to a hydrolysate containing lower toxin levels than the toxin levels in the hydrolysate prior to subjecting the hydrolysate to the multiple step detoxification process of the present disclosure, referred to herein as a “starting hydrolysate”. Such toxins include, but are not limited to, furan aldehydes, aliphatic acids, esters and phenolics.

Accordingly, the disclosure generally provides methods of reducing the toxicity of (i.e., detoxifying) a hydrolysate towards a fermenting organism. More particularly, the present disclosure relates to processes in which at least two different bases, or mixtures of bases, are added at different times to effectuate the detoxification of the hydrolysate. In certain aspects of the disclosure, detoxification involves a two step process. The first step involves mixing a starting solution of a hydrolysate (i.e., starting hydrolysate solution) with a first base or a first mixture of bases in an amount sufficient to raise the pH of the solution to a value sufficient to neutralize the majority of acids (e.g., aliphatic acids) present in the solution, and the second step involves mixing the hydrolysate solution with a second base or a second mixture of bases in an amount sufficient to raise the pH of the hydrolysate solution to a sufficient value and for a sufficient time to remove a substantial amount of toxins (e.g., furan aldehydes) in the solution, thereby producing a detoxified hydrolysate solution.

The first step of the hydrolysate detoxification process (i.e., mixing the hydrolysate with the first base or first mixture of bases) can be carried out at a pH ranging from 3 to 9, for example at a pH of 3, 4, 5, 6, 7, 8 or 9. In specific embodiments, the pH is in the range bounded by any of the two foregoing embodiments, e.g., a pH ranging from 3 to 4, from 3 to 5, from 4 to 6, etc.

The second step of the hydrolysate detoxification process (i.e., mixing the hydrolysate with the second base or second mixture of bases) can be carried out at a pH ranging from 7 to 10, for example at a pH of 7, 8, 9 or 10. In specific embodiments, the pH is in the range bounded by any of the two foregoing embodiments, e.g., a pH ranging from 7 to 9, from 8 to 9, from 8 to 10, etc.

The biomass is preferably lignocellulosic and can include, without limitation, seeds, grains, tubers, plant waste or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), wood (including, e.g., wood chips, processing waste), paper, pulp, and recycled paper (including, e.g., newspaper, printer paper, and the like). Other biomass materials include, without limitation, potatoes, soybean (e.g., rapeseed), barley, rye, oats, wheat, beets, and sugar cane bagasse. Further sources of biomass are disclosed in Section 4.1 and can be used in the present methods. Lignocellulosic hydrolysates obtained from a lignocellulosic biomass result in the production of sugars, aliphatic acids, phenolics, and products of sugar dehydration (e.g., furfural and 5-hydroxymethyl furfural (5-HMF)).

The concentration of the individual compounds of the hydrolysate in the hydrolysate solution prior to detoxification depends, in part, on the biomass from with the hydrolysate is obtained and the method used to hydrolyze the biomass. In certain embodiments, the starting hydrolysate solution comprises (a) total fermentable sugars at a concentration ranging from 30 g/L to 160 g/L, from 40 g/L to 95 g/L, or from 50 g/L to 70 g/L; (b) furfural at a concentration ranging from 0.5 g/L to 10 g/L, from 2.5 g/L to 4 g/L, or from 1.5 g/L to 5 g/L; (c) 5-HMF at a concentration ranging from 0.1 g/L to 5 g/L, from 0.5 g/L to 2.5 g/L or from 1 g/L to 2 g/L; (d) acetic acid at a concentration ranging from 2 g/L to 17 g/L or from 11 g/L to 16 g/L; (e) lactic acid at a concentration ranging from 0 g/L to 12 g/L or from 4 g/L to 10 g/L; (f) additional aliphatic acids (e.g., succinic acid, formic acid, butyric acid and levulinic acid) at concentrations ranging from 0 g/L to 2.5 g/L; and/or (g) phenolics at a concentration ranging from 0 g/L to 10 g/L, from 0.5 g/L to 5 g/L or from 1 g/L to 3 g/L.

The starting hydrolysate solution can be concentrated prior to detoxification. For instance, following biomass hydrolysis, a hydrolysate solution can be concentrated by 1.2-fold, 1.5-fold, 2-fold, 3-fold or 5-fold. In specific embodiments, the starting hydrolysate is concentrated in a range bounded by any two of the foregoing embodiments, e.g., concentrated in the range from 1-fold to 3-fold, 1.5-fold to 3-fold, 3-fold to 5-fold, etc.

Advantageously, the first base added to the hydrolysate solution comprises a magnesium base (e.g., magnesium hydroxide, magnesium carbonate or magnesium oxide), which can neutralize any acids present in the hydrolysate solution and react, to some extent, with other toxins in the hydrolysate solution. In particular embodiments, the first base added to the hydrolysate solution is magnesium hydroxide.

In certain aspects of the disclosure, the second base added to the hydrolysate solution includes ammonium hydroxide, sodium hydroxide, potassium hydroxide, calcium hydroxide, or mixtures thereof. In particular embodiments, the second base added to the hydrolysate solution is ammonium hydroxide.

In certain embodiments, each step of the detoxification process can be carried out at the same or at substantially similar temperatures. In these embodiments, detoxification of the hydrolysate solution can be carried out at a temperature of 25° C. or greater and 90° C. or lower. The detoxification process can be carried out, for example at 30° C., 35° C., 40° C., 45° C., 50° C., 55° C., 60° C., 65° C., 70° C., 75° C., 80° C., 85° C., or 90° C. In specific embodiments, the detoxification process can be carried out at a temperature in the range bounded by any two of the foregoing temperatures, e.g., at a temperature ranging from 40° C. to 60° C., from 45° C. to 55° C., from 45° C. to 50° C., etc.

In other embodiments, each step of the detoxification process can be carried out at different temperatures. For instance, the temperature of the hydrolysate solution following the addition of the first base can differ from the temperature of the hydrolysate solution following the addition of the second base and/or third base, and so forth. In embodiments involving additions of two bases (or two mixtures of bases), the temperature during the first step of the detoxification process (i.e., mixing the hydrolysate with the first base) can be in the range between 30° C. to 90° C., and more typically in the range between 40° C. to 70° C. In these embodiments, the temperature during the second step of the detoxification process (i.e., mixing the hydrolysate with the second base) can be in the range between 30° C. to 80° C., and more typically in the range between 40° C. to 60° C.

The detoxification process can be carried out as a batch process, as a continuous process, or as a semi-continuous process. For instance, the detoxification process can be carried out in a batch reactor, a continuous stirred tank reactor (CSTR) or a plug flow reactor (PFR). In certain embodiments, each step of the detoxification can be carried out in the same batch reactor. In these embodiments, the second base, or second mixture of bases can be added to the batch reactor after the acids in the starting hydrolysate solution have been sufficiently neutralized by the first base, or the first mixture of bases. In other embodiments, each step of the detoxification process can be carried out in different reactors. For instance, in particular embodiments involving the addition of two bases (or two mixtures of bases), the first step of the detoxification process (i.e., mixing the hydrolysate with the first base) can be carried in a CSTR and the second step (i.e., mixing the hydrolysate with the second base) can be carried out in a PFR. In other embodiments, the first step of the detoxification process can be carried out in a PFR and the second step can be carried out in a CSTR. In other embodiments, both steps can be carried out in a PFR. In still other embodiments, both steps can be carried out in a CSTR.

In certain embodiments, the disclosure provides methods for continuously reducing the quantity of furan aldehydes in a lignocellulosic hydrolysate, comprising the steps of flowing a hydrolysate solution into a first reactor or a first series of reactors, said solution comprising a mixture of fermentable sugars, furan aldehydes, phenolics and aliphatic acids, flowing a first base into the first reactor or the first series of reactors, mixing the hydrolysate solution with the first base in the first reactor or the first series of reactors for a period of time sufficient to neutralize acids in the hydrolysate solution, flowing the hydrolysate solution into a second reactor or a second series of reactors, flowing a second base into the second reactor or the second series of reactors, mixing the hydrolysate solution with the second base in the second reactor or the second series of reactors for a period of time sufficient to reduce the quantity of furan aldehydes in the hydrolysate solution, thereby producing a detoxified hydrolysate solution, and flowing the detoxified hydrolysate solution out of the second reactor or the second series of reactors.

The methods of the present disclosure provide a detoxified hydrolysate with at least 70%, at least 80%, at least 85%, at least 90%, at least 92%, at least 93%, at least 95% or at least 99% of the total fermentable sugars present in the starting hydrolysate and no greater than 70%, no greater than 60%, no greater than 50%, no greater than 40%, no greater than 30%, no greater than 20% or no greater than 10% of the furan aldehydes present in the staring hydrolysate. In particular embodiments, detoxification methods of the present disclosure provide a detoxified hydrolysate with (a) at least 90% of the total fermentable sugars present in the starting hydrolysate and no greater than 50% of the furan aldehyde present in the starting hydrolysate; (b) at least 90% of the total fermentable sugars present in the starting hydrolysate and no greater than 40% of the furan aldehydes present in the starting hydrolysate; (c) at least 90% of the total fermentable sugars present in the starting hydrolysate and no greater than 30% of the furan aldehydes present in the starting hydrolysate; (d) at least 90% of the total fermentable sugars present in the starting hydrolysate and no greater than 20% of the furan aldehydes present in the starting hydrolysate; (e) at least 80% of the total fermentable sugars present in the starting hydrolysate and no greater than 50% of the furan aldehydes present in the starting hydrolysate; (f) at least 80% of the total fermentable sugars present in the starting hydrolysate and no greater than 40% of the furan aldehydes present in the starting hydrolysate; (g) at least 80% of the total fermentable sugars present in the starting hydrolysate and no greater than 30% of the furan aldehydes present in the starting hydrolysate; or (h) at least 80% of the total fermentable sugars present in the starting hydrolysate and no greater than 20% of the furan aldehydes present in the starting hydrolysate.

The detoxified hydrolysates of the present disclosure can be more effectively fermented by a fermenting microorganism to produce fermentation products such as ethanol. Accordingly, the methods of the disclosure further include culturing a microorganism in the presence of a detoxified hydrolysate produced in accordance with the present disclosure under conditions in which a fermentation product is produced. Various fermenting microorganisms (e.g., ethanologens) can be used to produce ethanol, such as those described in Section 4.5.

### 3. BRIEF DESCRIPTION OF THE FIGURES AND TABLES

FIG. 1: Schematic of a flow diagram of an exemplary continuous process of the present disclosure.

FIG. 2: Graph depicting the amount of xylose and furfural elimination at different time points for detoxification reactions performed at different initial pH targets (pH 8.5 and 9.0) using a two step detoxification process with magnesium hydroxide followed by ammonium hydroxide.

FIG. 3: Schematic illustrating a two base detoxification process using a configuration of CSTRs in series. Each port could be used to deliver base or base slurry and each vessel can be held to temperatures independently.

### 4. DETAILED DESCRIPTION

The present disclosure relates to methods for detoxifying a hydrolysate obtained from a biomass and methods of producing a fermentation product such as ethanol from the detoxified hydrolysate. Types of biomass that can be used in the present methods include but are not limited to those described in Section 4.1. Methods of hydrolyzing the biomass are described in Section 4.2. Typical compositions of hydrolysates prior to detoxification are described in Section 4.3. Methods of detoxifying the hydrolysates using multiple bases are described in Section 4.4. Methods of fermenting the detoxified hydrolysate to produce fermentation products are described in Section 4.5 and methods of recovering the fermentation products are described in Section 4.6.

4.1. Biomass

The term “biomass,” as used herein, refers to any composition comprising cellulose (optionally also hemicellulose and/or lignin).

Relevant types of biomasses which can be hydrolyzed or detoxified according to the methods of the disclosure can include biomasses obtained from agricultural crops such as, e.g., containing grains; corn stover, grass, bagasse, straw e.g. from rice, wheat, rye, oat, barley, rape, sorghum; tubers. e.g., beet and potato.

The biomass is preferably lignocellulosic. The lignocellulosic biomass is suitably from the grass family. The proper name is the family known as Poaceae or Gramineae in the class Liliopsida (the monocots) of the flowering plants. Plants of this family are usually called grasses, and include bamboo. There are about 600 genera and some 9,000-10,000 or more species of grasses (Kew Index of World Grass Species).

Poaceae includes the staple food grains and cereal crops grown around the world, lawn and forage grasses, and bamboo.

The success of the grasses lies in part in their morphology and growth processes, and in part in their physiological diversity. Most of the grasses divide into two physiological groups, using the C3 and C4 photosynthetic pathways for carbon fixation. The C4 grasses have a photosynthetic pathway linked to specialized leaf anatomy that particularly adapts them to hot climates and an atmosphere low in carbon dioxide. C3 grasses are referred to as “cool season grasses” while C4 plants are considered “warm season grasses”.

Grasses may be either annual or perennial. Examples of annual cool season are wheat, rye, annual bluegrass (annual meadowgrass, Poa annua and oat). Examples of perennial cool season are orchardgrass (cocksfoot, Dactylis glomerata), fescue (Festuca spp.), Kentucky bluegrass and perennial ryegrass (Lolium perenne). Examples of annual warm season are corn, sudangrass and pearl millet. Examples of Perennial Warm Season are big bluestem, indiangrass, bermudagrass and switchgrass.

One classification of the grass family recognizes twelve subfamilies: These are 1) anomochlooideae, a small lineage of broad-leaved grasses that includes two genera (Anomochloa, Streptochaeta); 2) Pharoideae (aka Poaceae), a small lineage of grasses that includes three genera, including Pharus and Leptaspis; 3) Puelioideae a small lineage that includes the African genus Puelia; 4) Pooideae which includes wheat, barley, oats, brome-grass (Bromus) and reed-grasses (Calamagrostis); 5) Bambusoideae which includes bamboo; 6) Ehrhartoideae, which includes rice, and wild rice; 7) Arundinoideae, which includes the giant reed and common reed 8) Centothecoideae, a small subfamily of 11 genera that is sometimes included in Panicoideae; 9) Chloridoideae including the lovegrasses (Eragrostis, ca. 350 species, including teff), dropseed grasses (Sporobolus, some 160 species), finger millet (Eleusine coracana (L.) Gaertn.), and the muhly grasses (Muhlenbergia, ca. 175 species); 10) Panicoideae including panic grass, maize, sorghum, sugar cane, most millets, fonio and bluestem grasses; 11) Micrairoideae; 12) Danthoniodieae including pampas grass; with Poa which is a genus of about 500 species of grasses, native to the temperate regions of both hemisphere.

Agricultural grasses grown for their edible seeds are called cereals. Three common cereals are rice, wheat and maize (corn). Of all crops, 70% are grasses.

Therefore a preferred biomass is selected from the group consisting of the energy crops. In a further preferred embodiment, the energy crops are grasses. Preferred grasses include Napier Grass or Uganda Grass, such as Pennisetum purpureum; or, Miscanthus; such as Miscanthus giganteus and other varieties of the genus miscanthus, or Indian grass, such as Sorghastrum nutans; or, switchgrass, e.g., as Panicum virgatum or other varieties of the genus Panicum), giant reed (arundo donax), energy cane (saccharum spp.)., wood (including, e.g., wood chips, processing waste), paper, pulp, and recycled paper (including, e.g., newspaper, printer paper, and the like). In some embodiments the biomass is sugarcane, which refers to any species of tall perennial grasses of the genus Saccharum.

Other types of biomass include seeds, grains, tuber (e.g., potatoes and beets), plant waste or byproducts of food processing or industrial processing (e.g., stalks), corn and corn byproducts (including, e.g., corn husks, corn cobs, corn fiber, corn stover, and the like), wood and wood byproducts (including, e.g., processing waste, deciduous wood, coniferous wood, wood chips (e.g., deciduous or coniferous wood chips), sawdust (e.g., deciduous or coniferous sawdust)), paper and paper byproducts (e.g., pulp, mill waste, and recycled paper, including, e.g., newspaper, printer paper, and the like), soybean (e.g., rapeseed), barley, rye, oats, wheat, beets, sorghum sudan, milo, bulgur, rice, sugar cane bagasse, forest residue, agricultural residues, quinoa, wheat straw, milo stubble, citrus waste, urban green waste or residue, food manufacturing industry waste or residue, cereal manufacturing waste or residue, hay, straw, rice straw, grain cleanings, spent brewer's grain, rice hulls, salix, spruce, poplar, eucalyptus, Brassica carinata residue, Antigonum leptopus, sweetgum, Sericea lespedeza, Chinese tallow, hemp, rapeseed, Sorghum bicolor, soybeans and soybean products (soybean leaves, soybeans stems, soybean pods, and soybean residue), sunflowers and sunflower products (e.g., leaves, sunflower stems, seedless sunflower heads, sunflower hulls, and sunflower residue), Arundo, nut shells, deciduous leaves, cotton fiber, manure, coastal Bermuda grass, clover, Johnsongrass, flax, straw (e.g., barley straw, buckwheat straw, oat straw, millet straw, rye straw amaranth straw, spelt straw), amaranth and amaranth products (e.g., amaranth stems, amaranth leaves, and amaranth residue), alfalfa, and bamboo.

Yet further sources of biomass include hardwood and softwood. Examples of suitable softwood and hardwood trees include, but are not limited to, the following: pine trees, such as loblolly pine, jack pine, Caribbean pine, lodgepole pine, shortleaf pine, slash pine, Honduran pine, Masson's pine, Sumatran pine, western white pine, egg-cone pine, longleaf pine, patula pine, maritime pine, ponderosa pine, Monterey pine, red pine, eastern white pine, Scots pine, araucaria tress; fir trees, such as Douglas fir; and hemlock trees, plus hybrids of any of the foregoing. Additional examples include, but are not limited to, the following: eucalyptus trees, such as Dunn's white gum, Tasmanian blue gum, rose gum, Sydney blue gum, Timor white gum, and the E. urograndis hybrid; populus trees, such as eastern cottonwood, bigtooth aspen, quaking aspen, and black cottonwood; and other hardwood trees, such as red alder, Sweetgum, tulip tree, Oregon ash, green ash, and willow, plus hybrids of any of the foregoing.

4.2. Hydrolysis of Biomass

Any hydrolysis process can be used to prepare lignocellulosic hydrolysates, including acid hydrolysis and base hydrolysis. Acid hydrolysis is a cheap and fast method and can suitably be used. A concentrated acid hydrolysis is preferably operated at temperatures from 20° C. to 100° C., and an acid strength in the range of 10% to 45% (e.g., 10%, 10.5%, 11%, 11.5%, 12%, 12.5%, 13%, 13.5%, 14%, 14.5%, 15%, 15.5%, 16%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 37%, 37.5%, 38%, 38.5%, 39%, 39.5%, 40%, 41%, 41.5%, 42%, 42.5%, 43%, 43.5%, 44%, 44.5%, 45% or any range bounded by any two of the foregoing values). Dilute acid hydrolysis is a simpler process, but is optimal at higher temperatures (100° C. to 230° C.) and pressure. Different kinds of acids, with concentrations in the range of 0.001% to 10% (e.g., 0.001%, 0.01%, 0.05%, 0.1%, 0.15%, 0.2%, 0.25%, 0.3%, 0.35%, 0.4%, 0.5%, 0.6%, 0.7%, 0.8%, 0.9%, 1%, 2%, 3%, 4%, 5%, 5.5%, 6%, 6.5%, 7%, 7.5%, 8%, 8.5%, 9%, 9.5% or 10%, or any range bounded by any two of the foregoing values) are preferably used. Suitable acids including nitric acid, sulfurous acid, nitrous acid, phosphoric acid, acetic acid, hydrochloric acid and sulfuric acid can be used in the hydrolysis step. Preferably sulfuric acid is used.

Depending on the acid concentration, and the temperature and pressure under which the acid hydrolysis step is carried out, corrosion resistant equipment and/or pressure tolerant equipment may be needed.

The hydrolysis can be carried out for a time period ranging from 2 minutes to 10 hours (e.g., 2, 3, 4, 5, 6, 7, 8, 9, 10, 15, 20, 25, 26, 27, 28, 29, or 30 minutes, or 0.5, 0.75, 1, 1.5, 2, 2.5, 3, 3.5, 4, 4.5, 5, 5.5, 6, 6.5, 7, 7.5, 8, 8.5, 9, 9.5 or 10 hour, or range bounded by any two of the foregoing values), preferably 1 minute to 2 hours, 2 minutes to 15 minutes, 2 minutes to 2 hours, 15 minutes to 2 hours, 30 minutes to 2 hours, 10 minutes to 1.5 hours, or 1 hour to 5 hours.

The hydrolysis can also include, as an alternative (e.g., in the absence of) or in addition to (e.g., before or after) the acid treatment, a heat or pressure treatment or a combination of heat and pressure, e.g., treatment with steam, for about 0.5 hours to about 10 hours (e.g., 0.5, 1, 1.5, 2, 2.5, 3, 3.5, 4, 4.5, 5.5, 6, 6.5, 7, 7.5, 8, 8.5, 9, 9.5 or 10 hours, or any range bounded by any two of the foregoing values).

Variations of acid hydrolysis methods are known in the art and are encompassed by the methods of the present disclosure. For instance, the hydrolysis can be carried out by subjecting the biomass material to a two step process. The first (chemical) hydrolysis step is carried out in an aqueous medium at a temperature and a pressure chosen to effectuate primarily depolymerization of hemicellulose without achieving significant depolymerization of cellulose into glucose. This step yields slurry in which the liquid aqueous phase contains dissolved monosaccharides and soluble and insoluble oligomers of hemicellulose resulting from depolymerization of hemicellulose, and a solid phase containing cellulose and lignin. See, e.g., U.S. Pat. No. 5,536,325. In a preferred embodiment, sulfuric acid is utilized to affect the first hydrolysis step. After the sugars are separated from the first-stage hydrolysis process, the second hydrolysis step is run under harsher condition to hydrolyze the more resistant cellulose fractions.

In another embodiment, the hydrolysis method entails subjecting biomass material to a catalyst comprising a dilute solution of a strong acid and a metal salt in a reactor. The biomass material can, e.g., be a raw material or a dried material. This type of hydrolysis can lower the activation energy, or the temperature, of cellulose hydrolysis, ultimately allowing higher yields of fermentable sugars. See, e.g., U.S. Pat. Nos. 6,660,506; 6,423,145.

A further exemplary method involves processing a biomass material by one or more stages of dilute acid hydrolysis using about 0.4% to about 2% of an acid; followed by treating the unreacted solid lignocellulosic component of the acid hydrolyzed material with alkaline delignification. See, e.g., U.S. Pat. No. 6,409,841. Another exemplary hydrolysis method comprises prehydrolyzing biomass (e.g., lignocellulosic materials) in a prehydrolysis reactor; adding an acidic liquid to the solid lignocellulosic material to make a mixture; heating the mixture to reaction temperature; maintaining reaction temperature for a period of time sufficient to fractionate the lignocellulosic material into a solubilized portion containing at least about 20% of the lignin from the lignocellulosic material, and a solid fraction containing cellulose; separating the solubilized portion from the solid fraction, and removing the solubilized portion while at or near reaction temperature; and recovering the solubilized portion.

Hydrolysis can also comprise contacting a biomass material with stoichiometric amounts of sodium hydroxide and ammonium hydroxide at a very low concentration. See Teixeira et al., 1999, Appl. Biochem. and Biotech. 77-79:19-34. Hydrolysis can also comprise contacting a lignocellulose with a chemical (e.g., a base, such as sodium carbonate or potassium hydroxide) at a pH of about 9 to about 14 at moderate temperature, pressure, and pH. See PCT Publication WO 2004/081185.

Ammonia hydrolysis can also be used. Such a hydrolysis method comprises subjecting a biomass material to low ammonia concentration under conditions of high solids. See, e.g., U.S. Patent Publication No. 20070031918 and PCT publication WO 2006/110901.

Following hydrolysis, the hydrolyzed product comprises a mixture of acid or base, partially degraded biomass and fermentable sugars. Prior to further processing, the acid or base can be removed from the mixture by applying a vacuum. The mixture can also be neutralized prior to detoxification.

Prior to detoxification, the aqueous fraction comprising the solubilized sugars can be separated from insoluble particulates remaining in the mixture in a process referred to as solid/liquid separation. Methods for separating the soluble from the insoluble fractions include, but are not limited to, centrifugation (continuous, semi-continuous and batch), decantation and filtration. The hydrolyzed biomass solids can optionally be washed with an aqueous solvent (e.g., water) to remove adsorbed sugars.

The solids can be further processed prior to detoxification, for example dewatered. Dewatering can be suitably achieved with a screw press. The screw press is a machine that uses a large screw to pull a stream containing solids along a horizontal screen tube. Movement of the solids can be impeded by a weighted plate at the end of the tube. The pressure of this plate on the solid plug forces liquid out of the solids and through the holes in the sides of the screen tube and then along the effluent pipe. The screw will then push the remaining solids past the plate where they fall out onto a collection pad or conveyor belt below.

4.3. Hydrolysate Characteristics

Following hydrolysis and of the biomass the solid/liquid separation step, the lignocellulosic hydrolysate is subjected to detoxification. The relative amounts and concentrations of the individual compounds comprising the lignocellulosic hydrolysate solution prior to detoxification (i.e., starting lignocellulosic hydrolysate solution), including fermentable sugars, furan aldehydes, aliphatic acids and phenolics, are dependent on the particular lignocellulosic biomass and the hydrolysis method from which the hydrolysate was obtained.

In certain embodiments, the starting hydrolysate solution comprises (a) total fermentable sugars at a concentration ranging from 30 g/L to 160 g/L, from 40 g/L to 95 g/L, or from 50 g/L to 70 g/L; (b) furfural at a concentration ranging from 0.5 g/L to 10 g/L, from 2.5 g/L to 4 g/L, or from 1.5 g/L to 5 g/L; (c) 5-HMF at a concentration ranging from 0.1 g/L to 5 g/L, from 0.5 g/L to 2.5 g/L or from 1 g/L to 2 g/L; (d) acetic acid at a concentration ranging from 2 g/L to 17 g/L or from 11 g/L to 16 g/L; (e) lactic acid at a concentration ranging from 1 g/L to 12 g/L or from 4 g/L to 10 g/L; (f) additional aliphatic acids (e.g., succinic acid, formic acid, butyric acid and levulinic acid) at concentrations ranging from 0 g/L to 2.5 g/L; and/or (g) phenolics at a concentration ranging from 0 g/L to 10 g/L, from 0.5 g/L to 5 g/L or from 1 g/L to 3 g/L. In these embodiments, the starting hydrolysate solution will be referred to herein as “1×”.

In other embodiments, the starting hydrolysate can be more concentrated than 1×. For example, the starting hydrolysate solution can be 1.5-fold, 2-fold, 3-fold, 4-fold, 5-fold, 6-fold, 7-fold, 8-fold, 9-fold or 10-fold more concentrated than 1×. In these embodiments, the starting hydrolysate will be referred to as 1.5×, 2×, 3×, 4×, 5×, 6×, 7×, 8×, 9× and 10×, respectively.

In other embodiments, the starting hydrolysate can be less concentrated than 1×. For example, the starting hydrolysate solution can be 0.1-fold, 0.2-fold, 0.3-fold, 0.4-fold, 0.5-fold, 0.6-fold, 0.7-fold, 0.8-fold or 0.9-fold as concentrated as 1×. In these embodiments, the starting hydrolysate will be referred to as 0.1×, 0.2×, 0.3×, 0.4×, 0.5×, 0.6×, 0.7×, 0.8×, and 0.9, respectively.

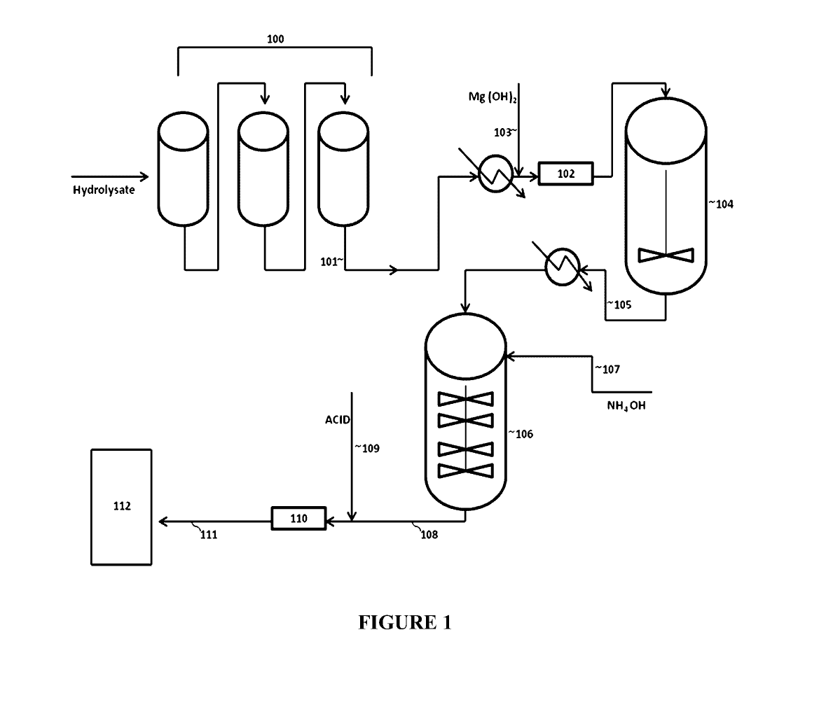

The concentration of the fermentable sugars and toxins can be adjusted prior to the detoxification process. Concentration of the hydrolysate solution can be particularly advantageous in the context of a continuous process (see FIG. 1 and Section 4.4). For example, a hydrolysate solution leaving a hydrolyzer following dilute acidic hydrolysis and solid/liquid separation can be concentrated prior to the addition of the first base used for detoxification. In certain embodiments, the hydrolysate solution can be concentrated by 1.2-fold, 1.5-fold, 2-fold, 3-fold or 5-fold prior to detoxification. In specific embodiments, the starting hydrolysate can concentrated in a range bounded by any two of the foregoing embodiments, e.g., concentrated by 1-fold to 3-fold, 1.5-fold to 3-fold, 3-fold to 5-fold, etc.

Concentrating the hydrolysate solution prior to detoxification can result in increased selectivity for furan aldehyde elimination over sugar degradation. Without being bound by theory, it is believed that the rate of reaction is first order with respect to sugar degradation and second order with respect to furan aldehyde elimination. Accordingly, concentrating the hydrolysate solution results in increasing the rate of elimination of furan aldehydes relative to the rate of degradation of fermentable sugars.

The hydrolysate solution can be concentrated under reduced pressure and/or by applying heat. In one embodiment, the hydrolysate solution is concentrated in a multi-stage evaporation unit (see FIG. 1 and Example 1). Concentration of hydrolysate can also be performed by other technologies such as membrane filtration, carbon treatment and ion-exchange resin. Evaporation results in increased sugar concentration and can result in the removal of some amounts of furfural and acetate.

4.4. Detoxification of Hydrolysates

The detoxification methods of the disclosure generally entail subjecting a lignocellulosic hydrolysate to a multiple step process in which at least two different bases or two different mixtures of bases are added at different times in the detoxification process. The detoxification methods are highly selective towards elimination of furan aldehydes. As used herein, the phrase “highly selective towards elimination of furan aldehydes” refers to the observation that furan aldehydes are eliminated (reacted) at higher rates than fermentable sugars are eliminated from the hydrolysate. As a result, the detoxified hydrolysates produced in accordance with the present disclosure have a larger percentage of fermentable sugars and a lower percentage of furan aldehydes relative to the starting hydrolysate. The detoxified lignocellulosic hydrolysates can then be fermented by a suitable fermenting microorganism (e.g., ethanologen) to produce a fermentation product (e.g., ethanol).

The detoxification methods typically comprise mixing a starting lignocellulosic hydrolysate solution with a first base, or a first mixture of bases, for a period of time and under conditions that result in the neutralization of the majority of the acids (e.g., aliphatic acids and counterions of acids used for hydrolysis) present in the hydrolysate solution, and then mixing the hydrolysate solution with a second base, or second mixture of bases, that substantially reduces the amount of toxins (e.g., furan aldehydes) in the hydrolysate solution. The bases used in each step can include, but are not limited to, magnesium hydroxide, magnesium carbonate, magnesium oxide, calcium hydroxide, ammonium hydroxide and sodium hydroxide. In certain embodiments, the first base, or first mixture of bases, is the same as the second base, or second mixture of bases. In other embodiments, the first base, or first mixture of bases, is different than the second base, or second mixture of bases.

The amount of time suitable to perform each step of the detoxification process depends on a number of factors, including the chemical composition of the hydrolysate, the concentration of the hydrolysate solution, the reaction temperature, the pH of the hydrolysate solution, the total amount of base added in each step, the stirring rate, and the type of reactor being used. In embodiments where the detoxification involves two steps, the first step of the detoxification can be carried out for a period of time ranging from 1 minute to 15 minutes and the second step of the detoxification can be carried out for a period of time ranging from 30 minutes to 20 hours. The overall detoxification process is typically carried out for a period of time ranging from 30 minutes to 20 hours, and more typically between 1 hour and 10 hours. For instance, the overall detoxification time can be 1 hour, 2 hours, 3 hours, 4 hours, 5 hours or 10 hours. In specific embodiments, the overall detoxification process is carried out for a period of time ranging from 1 hour to 6 hours, from 1.5 hours to 5 hours, from 2 hours to 5 hours, from 2.5 hours to 5 hours, from 2.5 hours to 4 hours, or from 3 hours to 4 hours.

The first step of the detoxification process (i.e., mixing with the first base or first mixture of bases) is typically carried out at a temperature of 95° C. or less, for example at a temperature of 30° C., 35° C., 40° C., 45° C., 50° C., 55° C., 60° C., 65° C., 70° C., 75° C., 80° C., 85° C., 90° C. or 95° C. In specific embodiments, the first step of the reaction can be carried out at a temperature bounded by any of the two foregoing embodiments, such as, but not limited to, a temperature ranging from 40° C. to 80° C., from 40° C. to 70° C., from 40° C. to 65° C., from 40° C. to 60° C., from 45° C. to 50° C., from 50° C. to 55° C., or from 40° C. to 50° C. The temperature of the mixture can be increased or decreased at any time following the addition of the hydrolysate solution.

In embodiments where the detoxification reaction involves two steps (i.e., two base additions), the second step of the process (i.e., mixing with the second base or second mixture of bases) is typically carried out at a temperature of 85° C. or less, for example at a temperature of 40° C., 45° C., 50° C., 55° C., 60° C., 65° C., 70° C., 75° C., 80° C., 85° C. or 90° C. The temperature of the first step of the detoxification process can be the same or different than the temperature of the second step of the detoxification process. In specific embodiments, the second step of the reaction can be carried out at a temperature bounded by any foregoing embodiments, such as, but not limited to, a temperature ranging from 40° C. to 80° C., from 40° C. to 70° C., from 40° C. to 65° C., from 40° C. to 60° C., from 40° C. to 50° C., from 50° C. to 55° C., from 45° C. to 50° C. or from 47° C. to 50° C. The temperature of the hydrolysate solution can be increased or decreased at any time following the addition of the second base.

In particular advantageous embodiments, the second step of the detoxification process is carried out at a temperature range between about 40° C. and 60° C., which allows the detoxification reactions to occur at a commercially feasible rate while minimizing the loss of fermentable sugars, and thereby increasing the yield of fermentation products (e.g., ethanol).

The first step of the hydrolysate detoxification process is typically carried out at a pH ranging from 3 to 9, for example at a pH of 3, 4, 5, 6, 7, 8 or 9. In specific embodiments, the pH is in the range bounded by any of the two foregoing embodiments, such as, but not limited to, a pH ranging from 3 to 4, from 3 to 5, from 4 to 5, or from 4 to 6. It will be understood that the pH of the hydrolysate solution following the addition of the first base depends on the nature and concentration of the base being added to the hydrolysate solution. The pH of the hydrolysate solution also depends on temperature. For instance, in embodiments in which the first base is magnesium hydroxide, the solubility of the magnesium hydroxide decreases with increasing temperature. Therefore, for a given amount of magnesium hydroxide added to the hydrolysate solution, the equilibrium pH decreases as the temperature is increased, all other variables being held constant.

The second step of the hydrolysate detoxification process is typically carried out at a pH ranging from 7 to 10, for example at a pH of 7, 8, 9 or 10. In specific embodiments, the pH is in the range bounded by any of the two foregoing embodiments, such as, but not limited to, a pH ranging from 7 to 8, from 7 to 9, from 8 to 9, or from 8 to 10. It will be understood that the pH of the hydrolysate solution following the addition of the second base depends on the nature and concentration of the base being added to the hydrolysate solution and the pH of the hydrolysate solution. It will be further understood that the pH of the solution can change as the detoxification process progresses. The pH of the hydrolysate solution can be adjusted during the process through the addition of a suitable acid or base.

In certain aspects of the disclosure, the first base that is added to the hydrolysate solution in the first step of the detoxification process is a magnesium base such as magnesium hydroxide, magnesium carbonate or magnesium oxide. The magnesium base can be added to the hydrolysate solution in a single step, multiple portions or continuously. In particular embodiments, the first base that is added to the hydrolysate solution is magnesium hydroxide. Magnesium hydroxide can be solubilized in aqueous solutions at acidic pH levels but has a low solubility at a neutral pH of 7 or higher. Hence, magnesium hydroxide provides a buffering effect near the equivalence point of the hydrolysate. A particular advantage of using magnesium hydroxide as the first base is that the buffering effect reduces the possibility of overshooting the pH prior to the addition of a second base or second mixture of bases in the second step of the detoxification process.

The total amount of magnesium base that is added to the hydrolysate solution in the first step of the detoxification process is depends on the desired pH of the hydrolysate solution. Higher pH levels can be obtained by adding larger amounts of magnesium base to achieve the desired pH. In embodiments where the magnesium base is magnesium hydroxide, the total amount of magnesium hydroxide that can be added to the hydrolysate solution 1× in the first step of the detoxification process to achieve a pH of between 3 and 8 can range from 1 gram per 1 kilogram hydrolysate (1 g/l kg hydrolysate) to 30 grams per 1 kilogram hydrolysate (30 g/l kg hydrolysate). For instance, the total amount of magnesium hydroxide added to the hydrolysate solution 1× can be 1 g/l kg hydrolysate, 2 g/l kg hydrolysate, 4 g/l kg hydrolysate, 6 g/l kg hydrolysate, 8 g/l kg hydrolysate, 10 g/l kg hydrolysate, 12 g/l kg hydrolysate, 15 g/l kg hydrolysate, 20 g/l kg hydrolysate or 25 g/l kg hydrolysate. In specific embodiments, the total amount of magnesium hydroxide added to the hydrolysate solution 1× is in the range bounded by any of the two foregoing embodiments, such as, but not limited to, magnesium hydroxide amounts ranging from 1 g/l kg hydrolysate to 20 g/l kg hydrolysate, from 1 g/l kg hydrolysate to 10 g/l kg hydrolysate, from 4 g/l kg hydrolysate to 20 g/l kg hydrolysate, from 4 g/l kg hydrolysate to 12 g/l kg hydrolysate, or from 10 g/l kg hydrolysate to 12 g/l kg hydrolysate. For more concentrated hydrolysate solutions (e.g., 4×), the amount of magnesium hydroxide sufficient to raise the pH to the desired level would be increased relative to hydrolysate solution 1×. For less concentrated hydrolysate solutions (e.g., 0.5×), the amount of magnesium hydroxide sufficient to raise the pH to the desired level would be decreased relative to hydrolysate solution 1×.

In certain aspects of the disclosure, the second base added to the hydrolysate solution includes ammonium hydroxide, sodium hydroxide, potassium hydroxide, calcium hydroxide, or mixtures thereof. In these embodiments, the pH of the solution is adjusted to between 7 and 11 following the addition of the second base or second mixture of bases. For instance, the pH of the hydrolysate following addition of the second base or second mixture of bases can be 7, 8, 9, 10 or 11. In specific embodiments, the pH of the solution following addition of the second base can be bounded by any two of the foregoing values, such as, but not limited to, a pH ranging from 7 to 9, 8 to 9, 8 to 10 or 9 to 11.

In certain aspects of the disclosure, the second base that is added to the hydrolysate solution in the second step of the detoxification process is ammonium hydroxide. Ammonium hydroxide has a pK_{b} of approximately 9.25. As a result, the pH of the hydrolysate solution can be elevated to the desired level (e.g., between 7 and 10, between 8 and 10, or between 8 and 9) without risk of overshooting the pH and thereby causing excess sugar degradation. Additionally, ammonium hydroxide provides a nitrogen source for a fermenting microorganism during fermentation (see Section 4.5.), which decreases the cost of the fermentation media. The total amount of ammonium hydroxide added to the hydrolysate solution 1× to bring the pH to the desired level can range from 1 grams per 1 kilogram hydrolysate (1 g/l kg hydrolysate) to 50 grams per 1 kilogram hydrolysate (50 g/l kg hydrolysate). For instance, the total amount of ammonium hydroxide added to the hydrolysate solution 1× can be 5 g/l kg hydrolysate, 10 g/l kg hydrolysate, 15 g/l kg hydrolysate, 20 g/l kg hydrolysate, 25 g/l kg hydrolysate, or 30 g/l kg hydrolysate. The ammonium hydroxide can be added to the hydrolysate solution in a single step, in multiple portions or continuously. In specific embodiments, the total amount of ammonium hydroxide added to the hydrolysate solution 1× is in the range bounded by any of the two foregoing embodiments, such as, but not limited to, ammonium hydroxide ranging from 1 g/l kg hydrolysate to 30 g/l kg hydrolysate, from 1 g/l kg hydrolysate to 20 g/l kg hydrolysate, from 1 g/l kg hydrolysate to 15 g/l kg hydrolysate, from 1 g/l kg hydrolysate to 10 g/l kg hydrolysate, or from 10 g/l kg hydrolysate to 22 g/l kg hydrolysate. For more concentrated hydrolysate solutions (e.g., 4×), the amount of ammonium hydroxide sufficient to raise the pH to the desired level would be increased relative to hydrolysate solution 1×. For less concentrated hydrolysate solutions (e.g., 0.5×), the amount of ammonium hydroxide sufficient to raise the pH to the desired level would be decreased relative to hydrolysate solution 1×.

In other aspects of the disclosure, the second base that is added to the hydrolysate solution in the second step of the detoxification process is calcium hydroxide. In these embodiments, removal of solid gypsum (calcium sulfate) by a belt filtration or centrifugation process is performed following detoxification. The total amount of ammonium hydroxide added to the hydrolysate solution 1× to bring the pH to the desired level (e.g., between 7 and 10, between 8 and 10, or between 8 and 9) can range from 2 grams per 1 kilogram hydrolysate (2 g/l kg hydrolysate) to 50 grams per 1 kilogram hydrolysate (50 g/l kg hydrolysate). For instance, the total amount of calcium hydroxide added to the hydrolysate solution 1× can be 2 g/l kg hydrolysate, 10 g/l kg hydrolysate, 15 g/l kg hydrolysate, 20 g/l kg hydrolysate, 25 g/l kg hydrolysate, or 30 g/l kg hydrolysate. The calcium hydroxide can be added to the hydrolysate solution in a single step, in multiple portions or continuously. In specific embodiments, the total amount of calcium hydroxide added to the hydrolysate solution 1× is in the range bounded by any of the two foregoing embodiments, such as, but not limited to, calcium hydroxide amounts ranging from 2 g/l kg hydrolysate to 30 g/l kg hydrolysate, from 2 g/l kg hydrolysate to 20 g/l kg hydrolysate, from 2 g/l kg hydrolysate to 15 g/l kg hydrolysate, from 2 g/l kg hydrolysate to 20 g/l kg hydrolysate, or from 10 g/l kg hydrolysate to 25 g/l kg hydrolysate. For more concentrated hydrolysate solutions (e.g., 4×), the amount of calcium hydroxide sufficient to raise the pH to the desired level would be increased relative to hydrolysate solution 1×. For less concentrated hydrolysate solutions (e.g., 0.5×), the amount of calcium hydroxide sufficient to raise the pH to the desired level would be decreased relative to hydrolysate solution 1×.

Each step of detoxification process of the present disclosure can be performed in any suitable vessel, such as a batch reactor or a continuous reactor (e.g., a continuous stirred tank reactor (CSTR) or a plug flow reactor (PFR)). A continuous reactor allows for continuous addition and removal of input materials (e.g., hydrolysate, magnesium base slurry) as the detoxification reaction progresses. The suitable vessel can be equipped with a means, such as impellers, for agitating the hydrolysate solution. Reactor design is discussed in Lin, K.-H., and Van Ness, H. C. (in Perry, R. H. and Chilton, C. H. (eds), Chemical Engineer's Handbook, 5th Edition (1973) Chapter 4, McGraw-Hill, NY).

The detoxification processes can be carried out in a batch mode. The methods typically involve combining the hydrolysate solution and the base (or base slurry) in the reactor. The hydrolysate solution and the detoxification base can be fed to the reactor together or separately. Any type of reactor can be used for batch mode detoxification, which simply involves adding material, carrying out the detoxification process at specified conditions (e.g. temperature, dosage and time) and removing the detoxified hydrolysate from the reactor.

Alternatively, the detoxification processes can be carried out in a continuous mode. The continuous processes of the disclosure advantageously reduces the need to stop and clean reactors and accordingly can be carried out in continuous mode, e.g., for periods of several days or longer (e.g., a week or more) to support an overall continuous process. The methods typically entail continuously feeding a reactor a hydrolysate solution and a base slurry. The hydrolysate and the base slurry can be fed together or separately. The resultant mixture has a particular retention or residence time in the reactor. The residence time is determined by the time to achieve the desired level of acid neutralization and/or detoxification following the addition of the hydrolysate and the base to the reactor. Following the detoxification process, the detoxified hydrolysate exits the reactor and additional components (e.g., hydrolysate and base slurry) can be added to the reactor. Multiple such reactors can be connected in series to support further pH adjustment during an extended retention time and/or to adjust temperature during an extended retention time.

For detoxification in continuous mode, any reactor can be used that allows equal input and output rates, e.g., a CSTR or PFR, so that a steady state is achieved in the reactor and the fill level of the reactor remains constant.

The detoxification processes of the disclosure can be carried out in semicontinuous mode. Semicontinuous reactors, which have unequal input and output streams that eventually require the system to be reset to the starting condition, can be used.

Each step of the detoxification can be carried out in the same reactor or in different reactors. For instance, in embodiments involving a two step detoxification process, the first and second step can be carried out in a batch reactor. In these embodiments, the second base, or second mixture of bases, is added after the first base, or first mixture of bases, neutralizes the acids present in the hydrolysate solution. The temperature of the batch reactor can be adjusted prior to or during the addition of the second base.

In certain embodiments involving a two step detoxification process, both the first step of the detoxification process (i.e., mixing the hydrolysate with the first base or first mixture of bases) and the second step of the detoxification process (i.e., mixing the hydrolysate with the second base or second mixture of bases) can be carried out in a CSTR (or a series of CSTRs) or PFR. In some embodiments, both the first step of the detoxification process and the second step of the detoxification process can be carried out in a CSTR (or a series of CSTRs). In other embodiments, both the first step of the detoxification process and the second step of the detoxification process can be carried out in a PFR. In still other embodiments, the first step of the detoxification process can be carried out in a CSTR (or a series of CSTRs) and the second step of the detoxification process can be carried out in a PFR. In still other embodiments, the first step of the detoxification process van be carried out in a PFR and the second step of the detoxification process can be carried out in a CSTR (or a series of CSTRs).

The methods of the disclosure can include further steps in addition to the multiple step detoxification process, such as one or more steps depicted in FIG. 1 that are upstream or downstream of the detoxification step. In FIG. 1, steps that are downstream of biomass hydrolysis are depicted. Following hydrolysis of the biomass and solid/liquid separation, the hydrolysate is concentrated in a multi-stage evaporation unit 100. The hydrolysate leaves the multi-stage evaporation unit 100 through line 101 and is pumped into mixer 102. A separate stream of magnesium hydroxide is pumped into mixer 102 through line 103. The mixture of the hydrolysate and the magnesium hydroxide is then pumped into CSTR 104. The residence time of the mixture in CSTR 104 is approximately 30 minutes to 1 hour. The pH in the CSTR is maintained in the range of between 5 and 6 and the temperature is in the range of between 45° C. and 60° C. The liquid stream exiting CSTR 104 is pumped into CSTR or PFR reactor 106 through line 105. Ammonium hydroxide is supplied continuously to the CSTR or PFR reactor 106 through line 107. The residence time of the mixture in the second CSTR or PFR reactor 106 is approximately 3 to 5 hours and the pH of the reactor is maintained in a range of between 8 and 10. Following detoxification in the second CSTR or PFR reactor 106, the detoxified hydrolysate is passed into line 108, where it is met with a stream of acid (e.g., sulfuric acid or phosphoric acid) from line 109. The mixture of detoxified hydrolysate is passed into mixer 110. The neutralized detoxified hydrolysate exits mixer 110 through line 111 and flows into fermentation vessel 112.

Adequate mixing of the hydrolysate solution following addition of each base, or mixture of bases, can improve the rate of dissolution of the base and ensure that the pH remains substantially homogeneous throughout the solution. For instance, ideal mixing will avoid the formation of local pockets of higher pH, which can result in lower selectivity for furan elimination. Mixing speeds of between 100 revolutions per minute (rpm) and 1500 rpm can be used to ensure sufficient mixing of the hydrolysate solution. For instance, mixing speeds of 100 rpm, 200 rpm, 400 rpm, 800 rpm and 1500 rpm can be used. In specific embodiments, mixing can be carried out at speeds bounded by any two of the foregoing mixing speeds, such as, but not limited to from 100 rpm to 200 rpm, from 100 rpm to 400 rpm, from 200 rpm to 400 rpm, from 400 rpm to 800 rpm or from 800 rpm to 1,500 rpm. In other embodiments, intermittent mixing regimes can be used where the rate of mixing is varied as the detoxification process progresses. Mixing of the hydrolysate solution can be accomplished using any mixer known in the art, such as a high-shear mixer, paddle mixer, magnetic stirrer or shaker, vortex, agitation with beads, and overhead stirring.

The detoxification methods of the present disclosure provide detoxified hydrolysates in which a substantial portion of the furan aldehydes (e.g., furfural) have been removed relative to the starting hydrolysate prior to detoxification. At the same time, the detoxification results in minimal loss of total fermentable sugars. Therefore, the detoxification reactions are highly selective towards elimination of furan aldehydes. In particular embodiments, the present disclosure provides a detoxified hydrolysate with at least 70%, at least 80%, at least 85%, at least 90%, at least 92%, at least 93%, at least 95% or at least 99% of the total fermentable sugars present in the starting hydrolysate and no greater than 50%, no greater than 40%, no greater than 30%, or no greater than 20% of the furan aldehydes present in the staring hydrolysate.

In particular embodiments, detoxification methods of the present disclosure provide a detoxified hydrolysate with (a) at least 90% of the total fermentable sugars present in the starting hydrolysate and no greater than 50% of the furan aldehyde present in the starting hydrolysate; (b) at least 90% of the total fermentable sugars present in the starting hydrolysate and no greater than 40% of the furan aldehydes present in the starting hydrolysate; (c) at least 90% of the total fermentable sugars present in the starting hydrolysate and no greater than 30% of the furan aldehydes present in the starting hydrolysate; (d) at least 90% of the total fermentable sugars present in the starting hydrolysate and no greater than 20% of the furan aldehydes present in the starting hydrolysate; (e) at least 80% of the total fermentable sugars present in the starting hydrolysate and no greater than 50% of the furan aldehydes present in the starting hydrolysate; (f) at least 80% of the total fermentable sugars present in the starting hydrolysate and no greater than 40% of the furan aldehydes present in the starting hydrolysate; (g) at least 80% of the total fermentable sugars present in the starting hydrolysate and no greater than 30% of the furan aldehydes present in the starting hydrolysate; or (h) at least 80% of the total fermentable sugars present in the starting hydrolysate and no greater than 20% of the furan aldehydes present in the starting hydrolysate.

After the detoxification process is complete, the pH of the detoxified hydrolysate solution can be lowered by adding a suitable acid (e.g., sulfuric acid or phosphoric acid) (see FIG. 1 and Example 3). The pH can be adjusted to a level that is suitable for a fermenting microorganism. Generally, the pH is adjusted to a value between 3.5 and 8, and more typically between a value of 4 and 7. After the pH is adjusted to the desired level, the detoxified hydrolysate can be transferred to a fermentation vessel.

4.5. Fermentation of Detoxified Hydrolysates

The fermentation of sugars to fermentation products can be carried out by one or more appropriate fermenting microorganisms in single or multistep fermentations. Fermenting microorganisms can be wild type microorganisms or recombinant microorganisms, and include Escherichia, Zymomonas, Saccharomyces, Candida, Pichia, Streptomyces, Bacillus, Lactobacillus, and Clostridium. Particularly suitable species of ethanologens include Escherichia coli, Zymomonas mobilis, Bacillus stearothermophilus, Saccharomyces cerevisiae, Clostridia thermocellum, Thermoanaerobacterium saccharolyticum, and Pichia stipitis. Genetically modified strains of E. coli or Zymomonas mobilis can be used for ethanol production (see, e.g., Underwood et al., 2002, Appl. Environ. Microbiol. 68:6263-6272 and US 2003/0162271 A1).

The fermentation can be carried out in a minimal media with or without additional nutrients such as vitamins and corn steep liquor (CSL). The fermentation can be carried out in any suitable fermentation vessel known in the art. For instance, fermentation can be carried out in an Erlenmeyer flask, Fleaker, DasGip fedbatch-pro (DasGip technology), 2 L BioFlo fermenter or 10 L fermenter (B. Braun Biotech) (see Example 5). The fermentation process can be performed as a batch, fed-batch or as a continuous process. The starting pH of the fermentation broth ranges from a value of 3.5 to a value of 8, and more typically from a value of 4 to a value of 7. The fermentation is generally carried out at a temperature between 20° C. and 40° C., and more typically between 25° C. and 35° C. In particular embodiments, the fermentation is carried out for a period of time between 5 to 90 hours, 10 to 50 hours, or from 20 to 40 hours.

4.6. Recovery of Fermentation Products

Fermentation products can be recovered using various methods known in the art. Products can be separated from other fermentation components by centrifugation, filtration, microfiltration, and nanofiltration. Products can be extracted by ion exchange, solvent extraction, or electrodialysis. Flocculating agents can be used to aid in product separation. As a specific example, bioproduced ethanol can be isolated from the fermentation medium using methods known in the art for ABE fermentations (see for example, Dune, 1998, Appl. Microbiol. Biotechnol. 49:639-648; Groot et al., 1992, Process. Biochem. 27:61-75; and references therein). For example, solids can be removed from the fermentation medium by centrifugation, filtration, decantation, or the like.

After fermentation, the fermentation product, e.g., ethanol, can be separated from the fermentation broth by any of the many conventional techniques known to separate ethanol from aqueous solutions. These methods include evaporation, distillation, azeotropic distillation, solvent extraction, liquid-liquid extraction, membrane separation, membrane evaporation, adsorption, gas stripping, pervaporation, and the like.

### 5. EXAMPLES

### 5.1. Example 1

### Hydrolysis of Lignocellulosic Biomass

A lignocellulosic biomass (e.g., energy cane or sugar cane) was harvested and sized using a forage chopper, inoculated with a preparation of Lactobacillus bacteria and stored in agricultural bags until use. Prior to dilute acid hydrolysis, the lignocellulosic biomass was removed from bags and washed with process water to remove organic acids and then dewatered with a screw press. The biomass was then conveyed to a pressurized reaction chamber (i.e., hydrolyzer) along with water and sulfuric acid (0.2% to 3%). The liquid/solid ratio of the slurry was minimized to maximize the dissolved sugar concentration in the hydrolysate following hydrolysis. The retention time in the hydrolyzer and the temperature of the hydrolyzer was dependent on parameters of the biomass (e.g., moisture and glucan levels). In general, the temperature of the hydrolyzer ranged from 120° C. to 180° C. and the retention time ranged from 3 minutes to 2 hours.

Following dilute-acid hydrolysis, the resultant hydrolyzer slurry contained solubilized sugars as well as residual insoluble fiber. The slurry was explosively decompressed and blown into a cyclone unit to depressurize the slurry. The material was reslurried with wash water and screw presses were used for dewatering the slurry in order to wring out soluble sugars and toxins. Three screw press steps with countercurrent washing were used to dewater and wash the cake of inhibitors. Countercurrent washing is defined as wash water flowing in the opposite direction to the cake flow. The high-percent solids slurry was diluted to a low percent solids slurry (<10% solids) and pumped to a screw press. This dilution was performed with a fraction of recycled liquids delivered by counter-current exchange from later screw presses (defined as “pressate”) as the system achieved steady-state. Clean water was added at the final screw press step along with the pressate to make the cake pumpable. The primary liquid/solid separation step was repeated with two more screw presses to remove toxins from the cake. The resulting high percent solids cake was carried forward for simultaneous saccharification and fermentation and the pressate from the first step was collected for detoxification work.

The concentrations of the individual compounds (e.g., sugars, furans and aliphatic acids) in the starting hydrolysate from several biomass sources following dilute acid hydrolysis and solid/liquid separation are shown in Table 1.

TABLE 1
Composition of Starting Hydrolysates

| | | | | | total | 5- | | succinic | lactic | formic | acetic | butyric | levulinic |
|---|---|---|---|---|---|---|---|---|---|---|---|---|---|
| | | glucose | xylose | Arabinose | sugar | HMF | Furfural | acid | acid | acid | acid | acid | acid |
| Name | Source | (g/L) | (g/L) | (g/L) | (g/L) | (g/L) | (g/L) | (g/L) | (g/L) | (g/L) | (g/L) | (g/L) | (g/L) |
| DP 110105 | Sugar | 22.74 | 67.05 | 6.83 | 96.61 | 1.04 | 3.13 | 0.00 | 2.04 | 1.37 | 15.55 | 0.00 | 1.03 |
| | Cane | | | | | | | | | | | | |
| DP 100309 | Sorghum | 14.4 | 42.55 | 7.00 | 63.95 | 0.36 | 3.08 | 0.22 | 10.92 | 0.53 | 11.53 | 0 | ND |
| DP 100511 | Energy | 11.01 | 46.6 | 6.25 | 63.86 | 0.33 | 1.7 | 0.00 | 6.85 | 0.43 | 9.83 | 0 | 0.49 |
| | Cane | | | | | | | | | | | | |
| DP 100513-1 | Energy | 10.75 | 57.27 | 8.55 | 76.56 | 0.36 | 2.73 | 0.33 | 10.06 | 0.50 | 11.59 | 0.00 | 0.61 |
| | Cane | | | | | | | | | | | | |
| DP 100513-2 | Energy | 15.1 | 54.2 | 7.95 | 77.25 | 0.42 | 2.97 | 0.064 | 12.00 | 0.66 | 14.34 | 0.00 | 0.61 |
| | Cane | | | | | | | | | | | | |

### 5.2. Example 2

### Two Step Detoxification of Sugar Cane Hydrolysate with Magnesium Hydroxide and Ammonium Hydroxide—Batch Processes

5.2.1. Materials and Methods

5.2.1.1. Sugar Cane DP 110105

Hydrolysate DP 110105, obtained from sugar cane, was placed in al L reactor vessel suitable for overhead stirring and heated to 47° C. by heating mantle and mixed vigorously. While the hydrolysate solution was warming, target amounts of magnesium hydroxide slurry (i.e., supersaturated solution of magnesium hydroxide in water) were weighed. The total quantity of magnesium hydroxide added to the hydrolysate was determined from the titration of the hydrolysate solution with sodium hydroxide. See Martinez et al., 2001, Biotechnol. Prog. 17(2):287-293. The magnesium hydroxide slurry was added to the hydrolysate solution at a dosage of 15.73 g/Kg hydrolysate at 47° C. while the solution was mixed vigorously and held for 5 minutes. The pH of the hydrolysate solution following addition of magnesium hydroxide was approximately 5.8.

Next, the pH of the hydrolysate solution was raised to between 8.3-8.7 by adding ammonium hydroxide at a dosage of 5.14 g/Kg hydrolysate. The progress of the detoxification process was monitored over time. Samples from the hydrolysate solution at various time points were taken and quenched with a stop solution (50 mM H₂SO₄) on ice (approximately 1.3 ml of each time point sample was immediately added to 11.7 ml of ice cold stop solution (50 mM H₂SO₄, 10× fold dilution) to quench any further reaction from occurring on the time scale of further chemical analysis). After the detoxification process was complete, the detoxified hydrolysate was then cooled to the fermentation temperature, and pH was adjusted to fermentation pH as described in Section 4.5. using 4M H₂SO₄ while mixing.

Following acidification of the detoxified hydrolysate solution, the concentrations of the individual compounds in the hydrolysate were measured. Sugars were separated and quantified by HPLC. A Shodex SP0810 size exclusion and ligand exchange column was used with an Agilent 1200 series refractive index detector (RID). An isocratic method was run using HPLC grade water as a mobile phase which provides enough resolution to generate a chromatogram from which the different sugar concentrations can be calculated, including xylose, arabinose, glucose, cellobiose, galactose, mannose, and other sugars.

Furfural and 5-HMF concentrations were also analyzed by HPLC using an Alltech Platinum C18 column and the same Agilent RID. Samples are diluted into a water/acetonitrile mixture and transferred into vials or well plate. These samples are identified and quantified by retention times and peak area against standard curves against known concentrations of various analytes.

5.2.1.2. Sugar Cane DP 110505

Hydrolysate DP 110505, obtained from sugar cane, was placed in a 2 L reactor vessel suitable for overhead stirring and heated to 47° C. by heating mantle and mixed vigorously. While the hydrolysate solution was warming, target amounts of magnesium hydroxide slurry (i.e., supersaturated solution of magnesium hydroxide in water) were weighed. The total quantity of magnesium hydroxide added to the hydrolysate was determined from the titration of the hydrolysate solution with sodium hydroxide. See Martinez et al., 2001, Biotechnol. Prog. 17(2):287-293. The magnesium hydroxide slurry was added to the hydrolysate solution at a dosage of 19.45 g/Kg hydrolysate at 47° C. while the solution was mixed vigorously and held for 5 minutes. The pH of the hydrolysate solution following addition of magnesium hydroxide was approximately 5.5.

Next, the pH of the hydrolysate solution was raised to between 8.3-8.7 by adding ammonium hydroxide at a dosage of 2.94 g/Kg hydrolysate. The progress of the detoxification process was monitored over time. Samples from the hydrolysate solution at various time points were taken and quenched with a stop solution (50 mM H₂SO₄) on ice (approximately 1.3 ml of each time point sample was immediately added to 11.7 ml of ice cold stop solution (50 mM H₂SO₄, 10× fold dilution) to quench any further reaction from occurring on the time scale of further chemical analysis). After the detoxification process was complete, the detoxified hydrolysate was then cooled to the fermentation temperature, and pH was adjusted to fermentation pH as described in Section 4.5. using 4M H₂SO₄ while mixing.

Following acidification of the detoxified hydrolysate solution, the concentrations of the individual compounds in the hydrolysate were measured as described in Section 5.2.1.1.

5.2.2. Results

Table 2 indicates the final pH values of the hydrolysate solution following addition of the first and second bases, the total reaction time, the % sugar loss measured after the detoxification process and the % furfural elimination measured after the detoxification process. The results shown in Table 2 indicate that detoxification reactions have far greater selectivity for furan aldehyde (e.g., furfural) elimination than for sugar loss. The percentage of sugar loss at the indicated time point was 0.8% or less, while the percentage of furfural removal was 33.2% or greater.

TABLE 2
Two Step Batch Detoxification of Sugar Cane with Magnesium Hydroxide and
Ammonium Hydroxide

| | | | | | | | Furfural destruction | |
|---|---|---|---|---|---|---|---|---|
| | | | | | | | % Furfural | |
| | | | | Final pH | | % Sugar Loss | Eliminated | |
| | | | Final pH | Following | | Following | Following | |
| | | | Following | Second | Reaction | Second | Second | |
| | | Hydrolysate | First Base | Base | Time | Detoxification | Detoxification | |
| Mixed Base | Biomass | Name | Addition | Addition | (hours) | Step | Step | Final furfural (g/L) |
| Mg(OH)₂/ | Sugar | DP 110105 | 5.8 | 8.7 | 5 | 0.0 | 55.7 | 1.13 |
| NH₄OH | cane | | | | | | | |
| | Sugar | DP 110505 | 5.5 | 8.43 | 3 | 0.0 | 33.6 | 1.67 |
| | cane | | | | | | | |

### 5.3. Example 3

### Two Step Batch Detoxification of Sugar Cane Hydrolysate DP 110505 with Magnesium Hydroxide and Ammonium Hydroxide—Effect of pH

5.3.1 Materials and Methods

Detoxification of DP 110105 from sugar cane, was carried out using a two step batch detoxification process with magnesium hydroxide and ammonium hydroxide in a similar fashion as described in Section 5.2.1.2. Experiments were run to measure the effect of pH following ammonium hydroxide on selectivity (furfural elimination vs. xylose degradation) at various time points.

Hydrolysate DP 110105 (800 g) was placed in a 1 L nonbaffled reactor vessel suitable for overhead stirring and heated to 47° C. by heating mantle and stirred at 420 rpm. After the hydrolysate solution was heated to the desired temperature, the magnesium hydroxide slurry was added rapidly to the hydrolysate solution to pH 5.8 at 47° C. and held for 5 minutes with stirring. Then ammonium hydroxide was added to a pH of either 8.5 or 9, and the mixture was stirred for a total of 4 hours at 47° C. The progress of the detoxification process was monitored over time. Samples from the hydrolysate solution at various time points were taken and quenched with a stop solution (50 mM H₂SO₄) on ice (approximately 1.3 ml of each time point sample was immediately added to 11.7 ml of ice cold stop solution (50 mM H₂SO₄, 10× fold dilution) to quench any further reaction from occurring on the time scale of further chemical analysis).

Following acidification of the detoxified hydrolysate solution, the concentrations of the individual compounds in the hydrolysate were measured. Sugars were separated and quantified by HPLC. A Shodex SP0810 size exclusion and ligand exchange column was used with an Agilent 1200 series refractive index detector (RID). An isocratic method was run using HPLC grade water as a mobile phase which provides enough resolution to generate a chromatogram from which the different sugar concentrations can be calculated, including xylose, arabinose, glucose, cellobiose, galactose, mannose, and other sugars.

Furfural and 5-HMF concentrations were also analyzed by HPLC using an Alltech Platinum C18 column and the same Agilent RID. Samples are diluted into a water/acetonitrile mixture and transferred into vials or well plate. These samples are identified and quantified by retention times and peak area against standard curves against known concentrations of various analytes.

5.3.2. Results

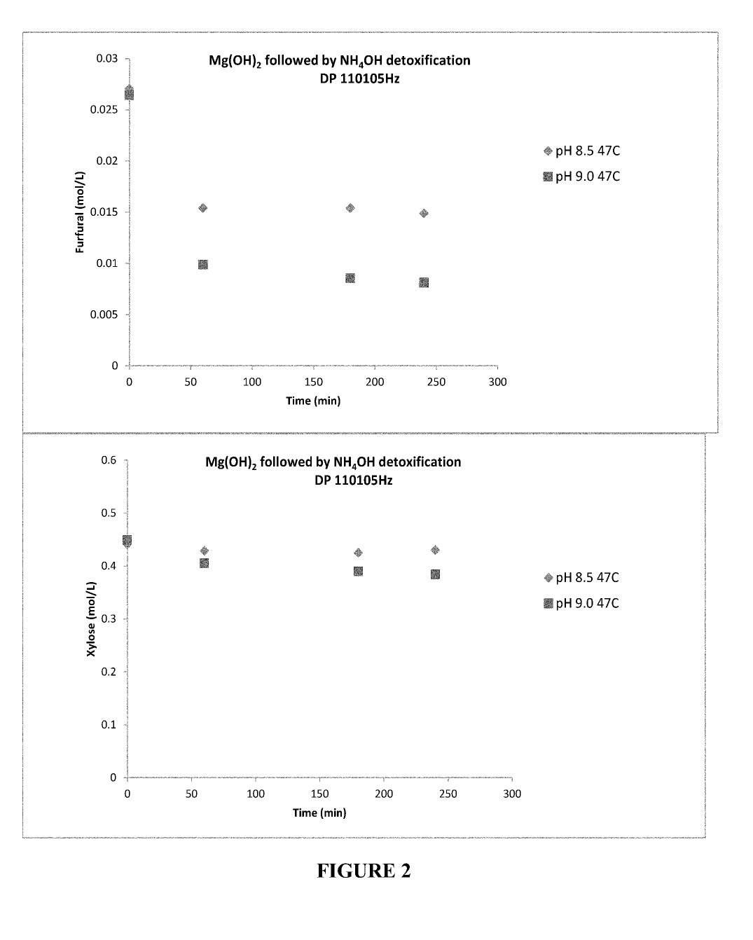

FIG. 2 depicts a graph illustrating the amount of furfural and xylose remaining at various times using the mixed base (magnesium hydroxide followed by ammonium hydroxide) detoxification procedure. As indicated in FIG. 2, the second step of the detoxification process was carried out at two different pH values (8.5 and 9). The results in FIG. 2 indicate that the detoxification process is highly selective at both a pH of 8.5 and 9. The rate of furfural elimination is faster at a pH of 9.

### 5.4. Example 4

### Two Step Detoxifications of Sugar Cane and Energy Cane

Hydrolysates with Magnesium Hydroxide and Calcium Hydroxide—Batch Process

5.4.1. Materials and Methods

5.4.1.1. Energy Cane Hydrolysate DP 100513-1

Hydrolysate DP 100513-1, derived from energy cane, was weighed in a 2 L round bottom flask equipped with a stir bar and preheated to 70° C. in an oil bath. While the hydrolysate solution was warming, target amounts of magnesium hydroxide slurry were weighed. The total quantity of magnesium hydroxide added to the hydrolysate was determined from the titration of the hydrolysate solution with sodium hydroxide. See Martinez et al., 2001, Biotechnol. Prog. 17(2):287-293. The magnesium hydroxide slurry was added to the hydrolysate solution at a dosage of 11.5 g/Kg hydrolysate at 70° C. while solution was mixed vigorously with a stir bar for approximately 5 minutes. The pH of the hydrolysate solution following addition of magnesium hydroxide was approximately 4.0.

Following the reaction with magnesium hydroxide, the hydrolysate solution was transferred to an empty beaker and cooled to 50° C. Next, the pH of the hydrolysate solution was raised to between 8.3-8.7 by adding calcium hydroxide at a dosage of 14.1 g/Kg hydrolysate while solution was mixed well by stir bar. Detoxified hydrolysate was then cooled to the fermentation temperature, and pH was adjusted to fermentation pH as described in Section 4.5. using 4M H₂SO₄ while mixing.

Following acidification of the detoxified hydrolysate solution, the concentrations of the individual compounds in the hydrolysate were measured as described in Section 5.2.1.1.

5.4.1.2. Sugar Cane Hydrolysate DP 110405

Hydrolysate DP 110405, derived from sugar cane, was weighed out in a 2 L round bottom flask equipped with a stir bar and preheated to 70° C. in an oil bath. While the hydrolysate solution was warming, target amounts of magnesium hydroxide slurry were weighed. The total quantity of magnesium hydroxide added to the hydrolysate was determined from the titration of the hydrolysate solution with sodium hydroxide. See Martinez et al., 2001, Biotechnol. Prog. 17(2):287-293. The magnesium hydroxide slurry was added to the hydrolysate solution at a dosage of 15.53 g/Kg hydrolysate at 70° C., while solution was mixed vigorously with a stir bar for approximately 5 minutes. The pH of the hydrolysate solution following addition of magnesium hydroxide was approximately 4.4.

Following the reaction with magnesium hydroxide, the hydrolysate solution was transferred to an empty beaker and cooled to 50° C. Next, the pH of the hydrolysate solution was raised to between 8.3-8.7 by adding calcium hydroxide at a dosage of 9.29 g/Kg hydrolysate. After calcium hydroxide addition, reaction was held for 6 hours to ensure sufficient detoxification. Detoxified hydrolysate was then cooled to the fermentation temperature, and pH was adjusted to fermentation pH as described in Section 4.5. using 4M H₂SO₄ while mixing.

Following acidification of the detoxified hydrolysate solution, the concentrations of the individual compounds in the hydrolysate were measured as described in Section 5.2.1.1.

5.4.1.3. Sugar Cane Hydrolysate DP 110105

Hydrolysate DP 110105, derived from sugar cane, was weighed out in a 2 L round bottom flask equipped with a stir bar and preheated to 70° C. in an oil bath. While the hydrolysate solution was warming, target amounts of magnesium hydroxide slurry were weighed. The total quantity of magnesium hydroxide added to the hydrolysate was determined from the titration of the hydrolysate solution with sodium hydroxide. See Martinez et al., 2001, Biotechnol. Prog. 17(2):287-293. The magnesium hydroxide slurry was added to the hydrolysate solution at a dosage of 11.84 g/Kg hydrolysate at 70° C., while solution was mixed vigorously with a stir bar for approximately 5 minutes. The pH of the hydrolysate solution following addition of magnesium hydroxide was approximately 4.4.

Following the reaction with magnesium hydroxide, the hydrolysate solution was transferred to an empty beaker and cooled to 50° C. Next, the pH of the hydrolysate solution was raised to between 8.3-8.7 by adding calcium hydroxide at a dosage of 8.55 g/Kg hydrolysate. After calcium hydroxide addition, reaction was held for 6 hours to ensure sufficient detoxification. Detoxified hydrolysate was then cooled to the fermentation temperature, and pH was adjusted to fermentation pH as described in Section 4.5. using 4M H₂SO₄ while mixing.

Following acidification of the detoxified hydrolysate solution, the concentrations of the individual compounds in the hydrolysate were measured as described in Section 5.2.1.

5.4.2. Results

Table 3 indicates the final pH values of the hydrolysate solution following addition of the first and second bases, the total reaction time, the % sugar loss measured after the detoxification process and the % furfural elimination measured after the detoxification process. The results shown in Table 3 indicate that detoxification reactions have far greater selectivity for furan aldehyde (e.g., furfural) elimination than for sugar loss. The percentage of sugar loss at the indicated time point was 5.0% or less, while the percentage of furan removal was 58.3% or greater.

TABLE 3
Two Step Batch Detoxification of Sugar Cane with Magnesium Hydroxide and
Calcium Hydroxide

| | | | | | | | Furfural destruction | |
|---|---|---|---|---|---|---|---|---|
| | | | | | | | % Furfural | |
| | | | | Final pH | | % Sugar Loss | Eliminated | |
| | | | Final pH | Following | | Following | Following | |
| | | | Following | Second | Reaction | Second | Second | |
| | | Hydrolysate | First Base | Base | Time | Detoxification | Detoxification | Final furfural |
| Mixed Base | Biomass | Name | Addition | Addition | (hours) | Step | Step | (g/L) |
| Mg(OH)₂/ | Sugar | DP 110405 | 4.36 | 8.81 | 6 | 5.0 | 58.3 | 0.98 |
| Ca(OH)₂ | cane | | | | | | | |
| | Sugar | DP 110105 | 4.51 | 8.71 | 6 | 4.0 | 60.6 | 1.14 |
| | cane | | | | | | | |
| | Energy | DP 110513 | 3.75 | 8.91 | 6 | 3.5 | 74.1 | 0.66 |
| | cane | | | | | | | |

### 5.5. Example 5

### Two Step Detoxification of Energy Cane Hydrolysate with Magnesium Hydroxide and Calcium Hydroxide—Series of CSTRs

5.5.1. Materials and Methods

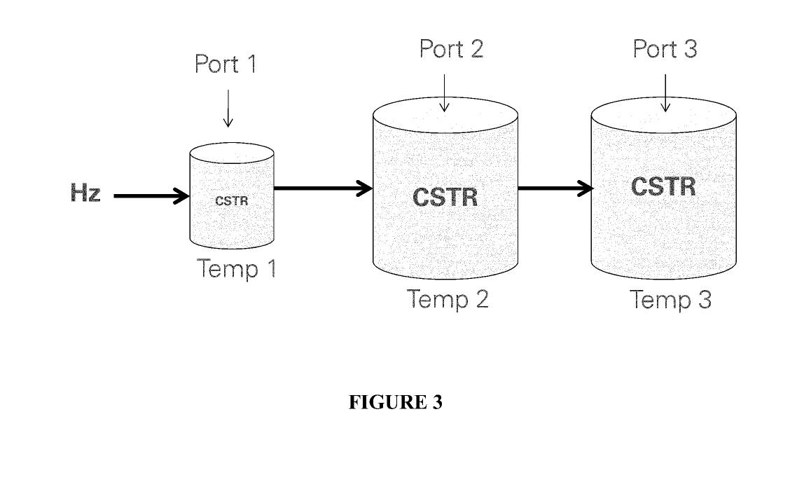

The detoxification of hydrolysate DP 100513-1, obtained from energy cane, was carried out using two individual CSTRs to perform each step of the detoxification process. Schematic overview is shown in FIG. 3. The hydrolysate solution (Hz) was heated to 70° C. with heating mantles and/or recirculating water bath and delivered by peristaltic pump to the first CSTR (250 ml) at a flow rate of 18.95 ml/min. The first CSTR was maintained at a temperature of 50° C. Magnesium hydroxide slurry (11.5 g/kg hydrolysate) was added to the first CSTR at a flow rate of 0.26 ml/min. The retention time in the first CSTR was constrained by fixing the target volume in each reactor flask and maintaining a target flow rate (where rate multiplied by volume equals the retention time). Hence, the retention time in the first reactor was approximately 3 minutes.

This mixture was then pumped to a second CSTR (2 L) to which calcium hydroxide (14.1 g/Kg hydrolysate) was added at a flow rate of 0.79 ml/min. The second CSTR was maintained at a temperature of 50° C. The retention time in the second CSTR was constrained by fixing the target volume in each reactor flask and maintaining a target flow rate (where rate multiplied by volume equals the retention time). Hence, the retention time in the second reactor was 1.7 hours. The mixture from the first reactor was pumped into the second CSTR (4 L) at a steady state flow rate which resulted in a total retention time of 3.3 hours.

To prevent build-up of foam, 100-fold diluted antifoam was added manually to the CSTR reactors at approximately 1 ml per hour.

5.5.2. Results

Table 4 indicates the final pH values of the hydrolysate solution following addition of the first and second bases, the total reaction time, the % sugar loss measured after the detoxification process and the % furfural elimination measured after the detoxification process carried in a CSTR. The results shown in Table 4 indicate that detoxification reactions have far greater selectivity for furan aldehyde (e.g., furfural) elimination than for sugar loss. The CSTR process results in detoxified hydrolysates with no sugar loss and greater than 87% furfural elimination.

TABLE 4
Two Step Detoxification of Sugar Cane with Magnesium Hydroxide and Calcium
Hydroxide in a CSTR

| | | | | | | Furfural destruction | |
|---|---|---|---|---|---|---|---|
| | | | | | | % Furfural | |
| | | | Final pH | | % Sugar Loss | Eliminated | |
| | | Final pH | Following | | Following | Following | |
| | | Following | Second | Reaction | Second | Second | |
| | Hydrolysate | First Base | Base | Time | Detoxification | Detoxification | Final furfural |
| Biomass | Name | Addition | Addition | (hours) | Step | Step | (g/L) |
| Energy | DP100513 | 4.06 | 8.79 | 5 | 0.0 | 87.5 | 0.3 |
| cane | | | | | | | |

### 5.6. Example 6

### Fermentation of Detoxified Hydrolysates

5.6.1. Materials and Methods

Following the detoxification step, pH of hydrolysate was adjusted to an appropriate fermentation pH (e.g., between 5 and 7) through the addition of 4M H₂SO₄ (see Examples 2 and 3). Fermentations of detoxified hydrolysate were conducted using E. coli and two different strains of S. cerevisiae (yeast) as ethanologens. Fermentation was carried out in minimal media with or without additional nutrient such as vitamins and CSL at starting pH between 5.0 and 7.0 with or without pH control and at a temperature between 32° C. to 35° C.

Processes include fermentation by Erlenmeyer flask, Fleaker (Spectrum Lab), DasGip fedbatch-pro (DasGip technology), 2 L BioFlo fermenter (New Brunswick), and 10 L fermenter (B. Braun Biotech). Batch and fed-batch fermentations have been tested in 2 L and 10 L fermenters. For example, E. coli inoculum cultures were grown in three steps. Seed I and II media consist of 40 mM MES, 1×AM6 (0.5 g/L sodium phosphate, 0.859 g/L urea), 1% CSL, and 60.79 g/L glucose. A 250 ml Erlenmeyer flask containing 100 ml medium was inoculated with 100 μl glycerol stock, and grown for 11 hours at 35° C. on a rotary shaker at 120 rpm (seed I). Seed II culture was inoculated with 100 μl of seed I culture, and grown for 11 hours at 35° C. on a rotary shaker at 120 rpm. Seed III culture containing 1×AM6, 5 g/L CSL, 50% detoxified hydrolysate (v/v), and 0.6% yeast autolysate was inoculated with 5% seed II culture in 2 L fermenter and grown at 35° C., pH7.0 with agitation at 495 rpm for 10-11 hrs until the ethanol concentration reached 5 g/L. The main fermentation vessel containing 95% (v/v) detoxified hydrolysate and 1×AM6 with or without additional nutrient was inoculated with 5% (v/v) seed III inoculum, and aerobic fermentation was carried out in both batch and fed-batch modes at 35° C. and at a pH of 7. During fed-batch fermentation, detoxified hydrolysate and AM6 were fed at various rates using a dissolved oxygen cascade control strategy by agitation ramping profile to maintain dissolved oxygen during feeding.

Ethanol concentrations from fermentation samples were determined using gas chromatography (GC, Agilent 6890 series). In particular, an Agilent system with a flame ionization detector and a HP-Innowax column was used. The GC system settings include 1) an HP-INNOWax polyethylene glycol capillary column (30 m×0.25 mm×0.25 um); 2) helium as carrier gas at 0.8 mL/min constant flow; 3) oven program: 40° C. (hold for 5.6 min), ramp 25C/min to 125° C.; 4) injection: inlet temperature 250° C., injection volume 1 uL with a split ratio of 100:1. The compound 1-propanol was used as internal standard and a multi-point standard curve was obtained to calculate the final ethanol concentration for each sample. Samples were diluted with methanol containing 0.2% 1-propanol as an internal standard and injected into GC system after removal of precipitates. Ethanol was identified by retention time and quantified by peak area.

5.6.2. Results

The ability of the ethanologen to manufacture ethanol, defined as fermentability, was assessed for detoxified hydrolysates following detoxification with magnesium hydroxide or with calcium hydroxide. Detoxification reactions with calcium hydroxide were run under standard overliming conditions (55° C. for 30 minutes) in similar fashion to detoxification reactions described in Examples 2 and 3.

The results for fermentability of the detoxified hydrolysates are shown in Table 5. In Table 5, the fermentability metric has been normalized to standard overliming conditions, where a fermentability of 1 is defined as a condition that reaches the same maximal ethanol concentration as the standard overliming condition. As shown in Table 5, the quantity of ethanol produced is comparable to that of hydrolysates that are detoxified with calcium hydroxide.

TABLE 5
Fermentability of Detoxified Hydrolysates

| | | | | Ethanol production | | |
|---|---|---|---|---|---|---|
| | | | | Fermentation | | |
| | | | | normalized to | Maximum ethanol | |
| | | Hydrolysate | Detoxification | overliming | production (g | |
| Mixed Base | Biomass | Name | Reactor | condition | EtOH/L/h) | Ethanologen |
| Mg(OH)₂/ | Sugar cane | DP110105 | Batch | 0.95 | 0.51 | S. cerevisiae |
| NH₄OH | | | | | | (strain 1) |
| | Sugar cane | DP110105 | CSTR | 0.77 | 0.42 | S. cerevisiae |
| | | | | | | (strain 1) |
| | Sugar cane | DP110105 | Batch | 0.92 | 0.39 | S. cerevisiae |
| | | | | | | (strain 2) |
| Mg(OH)₂/ | Sugar cane | DP110405 | Batch | NA | 0.34 | S. cerevisiae |
| Ca(OH)₂ | | | | | | (strain 2) |
| | Energy | DP100513 | CSTR | 0.99 | 0.90 | E. coli |
| | cane | | | | | |
| | Energy | DP100513 | Batch | 0.99 | 0.97 | E. coli |
| | cane | | | | | |

### 6. SPECIFIC EMBODIMENTS AND INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A method of reducing the toxicity of a lignocellulosic hydrolysate towards a fermenting organism, or for reducing at least a portion of one inhibitor to a fermenting organism from a lignocellulosic hydrolysate, comprising the steps of:
(a) mixing a starting solution of the lignocellulosic hydrolysate obtained from a lignocellulosic biomass, said starting solution comprising a mixture of fermentable sugars, furan aldehydes, and aliphatic acids, with a first base or a first mixture of bases in an amount sufficient to raise the pH of the solution to between 3 and 8; and
(b) mixing the solution produced in step (a) with a second base or a second mixture of bases in an amount sufficient to raise the pH of the solution to between 7 and 10 and for a time sufficient to eliminate at least 40% of the furan aldehydes in the lignocellulosic hydrolysate,
thereby reducing the toxicity of the lignocellulosic hydrolysate.

2. The method of claim 1, wherein the first base and the second base are the same.

3. The method of claim 1, wherein the first base and the second base are different.

4. The method of claim 1, wherein the first base is added in amount sufficient to raise the solution to a pH between 3 and 5.

5. The method of claim 1, wherein the first base is added in amount sufficient to raise the solution to a pH between 4 and 6.

6. The method of claim 1, wherein the second base is added in amount sufficient to raise the solution to a pH between 8 and 10.

7. The method of claim 1, wherein the second base is added in amount sufficient to raise the solution to a pH between 9 and 10.

8. The method of claim 1, wherein step (a) is carried out at a temperature of between 40° C. and 60° C.

9. The method of claim 1, wherein step (a) is carried out at a temperature of between 40° C. and 50° C.

10. The method of claim 1, wherein step (b) is carried out at a temperature of between 30° C. and 90° C.

11. The method of claim 1, wherein step (b) is carried out at a temperature of between 40° C. and 70° C.

12. The method of claim 1, wherein the lignocellulosic biomass is selected from Napier grass, energy cane, sorghum, giant reed, sugar beet, switchgrass, bagasse, rice straw, miscanthus, switchgrass, wheat straw, wood, wood waste, paper, paper waste, agricultural waste, municipal waste, birchwood, oat spelt, corn stover, eucalyptus, willow, hybrid poplar, short-rotation woody crop, conifer softwood and crop residue.

13. The method of claim 1, wherein the first base is a magnesium base.

14. The method of claim 13, wherein the magnesium base is magnesium hydroxide.

15. The method of claim 13, wherein the magnesium base is magnesium carbonate.

16. The method of claim 13, wherein the magnesium base is magnesium oxide.

17. The method of claim 1, wherein the second base is selected from ammonium hydroxide, calcium hydroxide, sodium hydroxide and potassium hydroxide.

18. The method of claim 17, wherein the second base is ammonium hydroxide.

19. The method of claim 17, wherein the second base is calcium hydroxide.

20. The method of claim 17, wherein the second base is sodium hydroxide.

21. The method of claim 17, wherein the second base is potassium hydroxide.

22. The method of claim 1, wherein step (a) and step (b) are carried out in a batch reactor.

23. The method of claim 1, wherein step (a) is carried out in a batch reactor and step (b) is carried out in a continuous stirred tank reactor (CSTR) or a series of CSTRs.

24. The method of claim 1, wherein both step (a) and step (b) are carried out in a CSTR or a series of CSTRs.

25. The method of claim 1, wherein step (a) is carried out in a CSTR or a series of CSTRs and step (b) is carried out in a plug flow reactor (PFR)

26. The method of claim 1, wherein both step (a) and step (b) are carried out in a PFR.

27. The method of claim 1, wherein mixing the starting hydrolysate with the magnesium base is carried out for a period of time between 0.05 hours and 4 hours.

28. The method of claim 1, wherein step (a) and step (b) are carried out for a combined period of time between 1 hour and 6 hours.

29. The method of claim 1, wherein step (a) and step (b) are carried out for a combined period of time between 2 hours and 5 hours.

30. The method of claim 1, wherein the concentration of total fermentable sugars in the starting solution is between 30 g/L and 160 g/L.

31. The method of claim 1, wherein the concentration of total fermentable sugars in the starting solution is between 40 g/L and 95 g/L.

32. The method of claim 1, wherein the concentration of total fermentable sugars in the starting hydrolysate is between 50 g/L and 70 g/L.

33. The method of claim 1, wherein the furan aldehydes are comprised of furfural and 5-HMF.

34. The method of claim 33, wherein the starting concentration of furfural in the starting solution is between 0.5 g/L and 10 g/L.

35. The method of claim 33, wherein the starting concentration of furfural in the starting solution is between 1.5 g/L and 5 g/L.

36. The method of claim 33, wherein the concentration of 5-HMF in the starting solution is between 0.1 g/L and 5 g/L.

37. The method of claim 33, wherein the concentration of 5-HMF in the starting solution is between 0.5 g/L and 2.5 g/L.

38. The method of claim 1, wherein the aliphatic acids are comprised of acetic acid and lactic acid.

39. The method of claim 38, wherein the concentration of acetic acid in the starting solution is between 2 g/L and 17 g/L.

40. The method of claim 38, wherein the concentration of acetic acid in the starting solution is between 11 g/L and 16 g/L.

41. The method of claim 38, wherein the concentration of lactic acid in the starting solution is between 4 g/L and 10 g/L.

42. The method of claim 1, wherein the starting hydrolysate further comprises phenolics.

43. The method of claim 42, wherein the concentration of phenolics in the starting solution is between 0.5 g/L and 5 g/L.

44. The method of claim 1, wherein the fermentable sugars include one or more of xylose, arabinose, rhamnose, glucose, mannose and galactose.

45. The method of claim 1, wherein the hydrolysate solution produced in step (b) comprises no greater than 30%, no greater than 20% or no greater than 10% of the furan aldehydes present in the starting lignocellulosic hydrolysate solution.

46. The method of claim 1, wherein the hydrolysate solution produced in step (b) comprises at least 90%, at least 93% or at least 95% of the total fermentable sugars present in the starting hydrolysate solution.

47. The method of claim 1, further comprising the step of adding an acid to lower the pH of the solution produced in step (b) to between 3.5 and 9.

48. The method of claim 1, further comprising the step of adding an acid to lower the pH of the solution produced in step (b) to between 4 and 6.

49. The method of claim 1, further comprising the step of concentrating the starting hydrolysate solution prior to step (a).

50. A method of producing ethanol, comprising the step of culturing a fermenting microorganism in the presence of a detoxified hydrolysate solution produced by the method of claim 1 under conditions in which ethanol is produced, thereby producing ethanol.

51. The method of claim 50, further comprising separating the ethanol from the culture.

52. The method of claim 50, wherein the fermenting organism includes one or more of Escherichia coli, Zymomonas mobilis, Bacillus stearothermophilus, Saccharomyces cerevisiae, Clostridia thermocellum, Thermoanaerobacterium saccharolyticum, and Pichia stipitis.

53. The method of claim 50, further comprising producing the detoxified hydrolysate prior to said culturing step.

54. A method for continuously reducing the quantity of furan aldehydes in a lignocellulosic hydrolysate, comprising the steps of:
(a) flowing a hydrolysate solution into a first reactor or a first series of reactors, said hydrolysate solution comprising a mixture of fermentable sugars, furan aldehydes, and aliphatic acids;
(b) flowing a first base into the first reactor or the first series of reactors;
(c) mixing the hydrolysate solution with the first base in the first reactor or the first series of reactors for a period of time sufficient to neutralize the acids in the hydrolysate solution;
(d) flowing the hydrolysate solution into a second reactor or the second series of reactors;
(e) flowing a second base into the second reactor or the second series of reactors;
(f) mixing the hydrolysate solution with the second base in the second reactor or the second series of reactors for a period of time sufficient to reduce the quantity of furan aldehydes in the hydrolysate, thereby producing a detoxified hydrolysate solution; and
(g) flowing the detoxified hydrolysate solution out of the second reactor or the second series of reactors.

55. The method of claim 54, further comprising acidifying the hydrolysate flowing out of the second reactor.

56. The method of claim 54, wherein the first base is a magnesium base.

57. The method of claim 56, wherein the magnesium base is selected from magnesium hydroxide, magnesium oxide and magnesium carbonate.

58. The method of claim 57, wherein the first base is magnesium hydroxide.

59. The method of claim 54, wherein the second base is ammonium hydroxide.

60. The method of claim 54, wherein the second base is calcium hydroxide.

61. The method of claim 54, wherein the first reactor is a CSTR and the second reactor is a PFR.

62. The method of claim 54, wherein the first reactor is a PFR and the second reactor is a plug flow reactor PFR.

63. The method of claim 54, wherein the first reactor is a PFR and the second reactor is a CSTR.

64. The method of claim 54, wherein the first reactor is a CSTR and the second reactor is a CSTR.

65. The method of claim 54, further comprising concentrating the hydrolysate prior to step (a).
